(19) United States
(12) Patent Application Publication
(10) Pub. No.: US 2022/0081393 A1
(43) Pub. Date: Mar. 17, 2022

(54) PROCESS FOR PREPARING ETHYLENICALLY UNSATURATED COMPOUNDS CONTAINING URETHANE GROUPS

(71) Applicant: Evonik Operations GmbH, Essen (DE)

(72) Inventors: Emmanouil Spyrou, Schermbeck (DE); Holger Loesch, Herne (DE); Susanne Kreischer, Herten (DE); Andrea Diesveld, Gescher (DE); Andrea Thesing, Ahaus (DE)

(21) Appl. No.: 17/472,468

(22) Filed: Sep. 10, 2021

(30) Foreign Application Priority Data
Sep. 11, 2020 (EP) 20195677.8

(51) Int. Cl. C07C 269/02; C08G 18/10; C08G 18/34; C08G 18/75; C08G 18/24; C08G 18/48

(52) U.S. Cl. CPC C07C 269/02; C08G 18/10; C08G 18/4825; C08G 18/755; C08G 18/246; C08G 18/348

(57) ABSTRACT

The present invention relates to a process for preparing at least one ethylenically unsaturated, urethane group-containing compound from
i) diisocyanate,
ii) at least one compound having at least one hydroxyl group and at least one ethylenically unsaturated, polymerizable group,
iii) and optionally at least one hydroxyl group-containing compound,
in which
1 diisocyanate is subjected to a determination of the peroxide content immediately prior to the reaction,
2 a) if the determined peroxide content is greater than or equal to 20 mmol/kg, the diisocyanate is subjected to distillative purification until the determined peroxide content is less than 20 mmol/kg, or
   or
   b) if the determined peroxide content is less than 20 mmol/kg, no distillative purification is performed, and
3. then i) distilled diisocyanate of a) and/or untreated diisocyanate of b) is reacted with ii) at least one compound having at least one hydroxyl group and at least one ethylenically unsaturated, polymerizable group and optionally iii) with at least one hydroxyl group-containing compound to give an ethylenically unsaturated, urethane group-containing compound.

The present invention relates to a process for preparing at least one ethylenically unsaturated, urethane group-containing compound from diisocyanate and at least one hydroxyl group-containing compound having ethylenically unsaturated, polymerizable groups.

Urethane group-containing compounds with ethylenically unsaturated groups play an important role in radiation-curing coating compositions.

For instance, U.S. Pat. No. 4,260,703 A discloses ester diol urethane acrylates which can be used in coating compositions alone or together with other constituents such as solvents, reactive oligomers and monomers, crosslinkers, fillers or other additives.

WO 2017/151387 A1 discloses a process for preparing a curable resin composition in which an isocyanate, a polyol mixture and a capping agent, which may be a hydroxyalkyl acrylate or methacrylate, are reacted. The resulting resin compositions can be used to produce composites, coatings, adhesives, printing inks, encapsulations and cast components.

To date, the ethylenically unsaturated, urethane group-containing compounds prepared or the precursors thereof have had the disadvantage of a tendency toward undesired premature polymerization (gelling). There is thus an interest in preventing this premature polymerization.

Surprisingly, it has been found that in particular in relatively old batches of ethylenically unsaturated, urethane group-containing compounds there are peroxide impurities present which lead to the mentioned gelling. It has furthermore been found that the undesired premature polymerization/gelling does not occur when there is a peroxide content of less than 20 mmol/kg in the diisocyanate batches used.

The present invention thus provides a process for preparing at least one ethylenically unsaturated, urethane group-containing compound from i) diisocyanate, ii) at least one compound having at least one hydroxyl group and at least one ethylenically unsaturated, polymerizable group, and iii) optionally at least one hydroxyl group-containing compound, in which
1. diisocyanate is subjected to a determination of the peroxide content immediately prior to the reaction,
2. a) if the determined peroxide content is greater than or equal to 20 mmol/kg, the diisocyanate is subjected to distillative purification until the determined peroxide content is less than 20 mmol/kg, or
   or
   b) if the determined peroxide content is less than 20 mmol/kg, no distillative purification is performed, and
3. then i) distilled diisocyanate of a) and/or untreated diisocyanate of b) is reacted with ii) at least one compound having at least one hydroxyl group and at least one ethylenically unsaturated, polymerizable group and optionally iii) with at least one hydroxyl group-containing compound to give an ethylenically unsaturated, urethane group-containing compound.

The present invention provides a process for preparing at least one ethylenically unsaturated, urethane group-containing compound from diisocyanate and at least one compound having at least one hydroxyl group and at least one ethylenically unsaturated, polymerizable group. The “diisocyanate” reactant may be a single diisocyanate or a mixture of diisocyanates. The reactant is preferably exactly one diisocyanate.

Preferably, at least one of the diisocyanates used is a (cyclo)aliphatic diisocyanate, that is to say a diisocyanate having at least one isocyanate group bonded directly to an aliphatic ring and possibly a further aliphatically bonded isocyanate group (that is to say one joined to the aliphatic ring via an alkylene radical). More preferably, only one (cyclo)aliphatic diisocyanate is used. Very particularly preferably, at least one of the diisocyanates used is isophorone diisocyanate (IPDI) or 4,4′-diisocyanatodicyclohexylmethane (H12MDI). More preferably still, isophorone diisocyanate is used as the sole diisocyanate. If isophorone diisocyanate is used, it is unimportant whether it has been obtained via the urea process or via the phosgene process.

The compound having at least one hydroxyl group and at least one ethylenically unsaturated, polymerizable group can in principle be any compound having at least one hydroxyl group and at least one ethylenically unsaturated, polymerizable group. It preferably has exactly one hydroxyl group and exactly one ethylenically unsaturated group.

Preferred compounds having at least one hydroxyl group and at least one ethylenically unsaturated, polymerizable group may be selected from the group consisting of hydroxyalkyl acrylates, hydroxyalkyl methacrylates and hydroxyalkyl vinyl ethers. These are preferably compounds wherein the hydroxyalkyl groups have C₂-C₁₀ alkylene radicals, preferably linear C₂-C₁₀ alkylene radicals. Very particular preference is given to hydroxyethyl acrylate, hydroxyethyl methacrylate, hydroxypropyl acrylate and hydroxypropyl methacrylate.

The partial or complete reaction of diisocyanate with the at least one compound having at least one hydroxyl group and at least one ethylenically unsaturated, polymerizable group is preceded by a determination of the peroxide content in the diisocyanate to be used. The peroxide content is determined according to DIN EN ISO 27107 and is determined in mmol/kg.

“Preceding” the reaction is preferably understood in this case to mean a time window of 14 days to 5 minutes before the mixing of diisocyanate and at least one reactant. It is very particularly preferably understood to mean a time of 2 days (48 h) prior to the reaction.

If only one diisocyanate is used, the peroxide content of the diisocyanate to be used is the content of peroxide in mmol based on the total mass of the diisocyanate to be used in kilograms. If more than one diisocyanate is used, the peroxide content of the diisocyanate to be used is the total content of peroxide in mmol based on the total mass of all diisocyanates to be used in kilograms.

If the peroxide content, determined as defined hereinabove, is less than 20 mmol/kg, no further action is taken, since no appreciable disadvantages due to the presence of such a concentration of peroxide are to be expected. The reactants can thus be reacted directly.

However, if the peroxide content, determined as defined hereinabove, is greater than or equal to 20 mmol/kg, the diisocyanate reactant is subjected to distillative purification. If only one diisocyanate is used, each batch of the diisocyanate having a peroxide content of greater than or equal to 20 mmol/kg is purified by distillation until the peroxide content of each batch is less than 20 mmol/kg. If the intention is to use two or more diisocyanates, each batch of each diisocyanate having a peroxide content of greater than or equal to 20 mmol/kg is purified by distillation until the peroxide content of each batch of each diisocyanate is less than 20 mmol/kg.

The distillative purification is preferably conducted in suitable distillation columns or distillation units, for example short-path or thin-film distillation apparatuses, at suitable pressures and temperatures depending on the boiling temperature of the diisocyanates. The minimum distillation temperature should in this case preferably not be below 100° C.

In addition to diisocyanate i) and at least one compound having at least one hydroxyl group and at least one ethylenically unsaturated, polymerizable group ii), it is optionally additionally possible to use iii) at least one hydroxyl group-containing compound in the preparation of the at least one ethylenically unsaturated compound. If this is the case, it is possible to prepare ethylenically unsaturated, urethane group-containing compounds having advantageous properties. Compound iii) does not have any ethylenically unsaturated groups.

Preferred hydroxyl group-containing compounds iii) can be selected from the group consisting of diols and polyols. Polyols are understood to be hydroxyl group-containing compounds having (on average) more than two hydroxyl groups. The diols or polyols can be monomeric, oligomeric or polymeric. Preferred oligomeric and polymeric diols and polyols have an OH number of 5 to 500 mg KOH/g (determined in accordance with DIN EN ISO 4692-2). Preferred diols and polyols are aliphatic diols and polyols, polyester diols and polyols, and polyether diols and polyols.

The hydroxyl group-containing compounds iii) used are preferably diols.

The reaction of i) diisocyanate, ii) compound having at least one hydroxyl group and at least one ethylenically unsaturated, polymerizable group and optionally iii) hydroxyl group-containing compound is preferably conducted in the presence of at least one catalyst at temperatures of 0-160° C. The pressure is not set specially in this case and corresponds to ambient pressure, which is close to 1 bar. Preferred reaction temperatures are 40-140° C. and more preferably still 60-90° C.

Preferred reaction times are between 3 minutes and three hours.

Suitable catalysts are the catalysts customary in polyurethane chemistry. Preferred catalysts can be selected from the group consisting of aminic catalysts (preferably diazabicyclooctane) and transition metal-based catalysts. Particularly preferred transition metal-based catalysts are dibutyltin laurate, bismuth neodecanoate, zinc octoate and zirconium acetylacetonate.

The catalyst is preferably used in amounts of from 0.001%-1.5% by weight, more preferably in amounts of from 0.01%-0.8% by weight, more preferably still 0.05%-0.1% by weight, based on the mass of the components used.

Although solvents (especially acetone, ethyl acetate, butyl acetate and methoxypropyl acetate) may in principle also be used, preference is given to working without solvent.

The process according to the invention can be carried out either batchwise or continuously. It is preferably carried out in a batch process.

If at least one diisocyanate i), at least one compound having at least one hydroxyl group and at least one ethylenically unsaturated, polymerizable group ii), and at least one hydroxyl group-containing compound iii) are used in the process according to the invention, all reactants i), ii) and iii) can in principle be reacted with each other simultaneously. However, it is very particularly advantageous to first react diisocyanate i) with at least one compound having at least one hydroxyl group and at least one ethylenically unsaturated, polymerizable group ii) and only thereafter to add at least one hydroxyl group-containing compound iii).

However, it may also be advantageous, for other reasons, to first react diisocyanate i) with at least one hydroxyl group-containing compound iii) and only thereafter to add at least one compound having at least one hydroxyl group and at least one ethylenically unsaturated, polymerizable group ii) (prepolymer mode).

Particularly good ethylenically unsaturated, urethane group-containing compounds additionally result when, even if the determined peroxide content is greater than or equal to 10 mmol/kg, the diisocyanate is subjected to distillative purification until the determined peroxide content is less than 10 mmol/kg. More preferably, even if the determined peroxide content is greater than or equal to 1 mmol/kg, the diisocyanate is subjected to distillative purification until the determined peroxide content is less than 1 mmol/kg.

It is additionally advantageous when the diisocyanate(s) has/have at least 0.1 mmol/kg of peroxide, i.e. each batch has at least 0.1 mmol/kg of peroxide.

More preferably, the process according to the invention is also conducted such that in step 2.

a) if the determined peroxide content is greater than 10 mmol/kg, the diisocyanate is subjected to distillative purification until the determined peroxide content is less than or equal to 10 mmol/kg but greater than or equal to 0.1 mmol/kg,
or if the determined peroxide content is less than 0.1 mmol/kg, peroxide is added until the peroxide content is greater than or equal to 0.1 mmol/kg but less than or equal to 10 mmol/kg, or
b) if the determined peroxide content is less than or equal to 10 mmol/kg but greater than or equal to 0.1 mmol/kg, no further action is taken.

### EXAMPLES

### Inventive Example

139 g of hydroxyethyl acrylate (1.2 mol, Aldrich) are added dropwise within 30 min to a mixture of 222 g of isophorone diisocyanate having a peroxide content of <10 mmol/kg (IPDI, 1 mol, Evonik Industries) and 0.7 g of dibutyltin dilaurate, and the mixture is then heated to 70° C. for a further 1 h to complete the reaction. 390 g of Poly THF 1000 (DuPont, polyether diol, OH number 115) are then added dropwise at this temperature within 30 min and the mixture is then again kept at 70° C. for 1 h. The NCO number thereafter was <0.1%. The reaction product (urethane acrylate) is liquid and colourless.

### Comparative Example

139 g of hydroxyethyl acrylate (1.2 mol, Aldrich) are added dropwise within 30 min to a mixture of 222 g of isophorone diisocyanate having a peroxide content of 110 mmol/kg (IPDI, 1 mol, Evonik Industries, into which dry air had been injected at 80° C. for three days) and 0.7 g of dibutyltin dilaurate, and the mixture is then heated to 70° C. for a further 1 h to complete the reaction. 390 g of Poly THF 1000 (DuPont, polyether diol, OH number 115) are then added dropwise at this temperature within 30 min and the mixture is then again kept at 70° C. for 1 h. The NCO number thereafter was <0.1%. The reaction product (urethane acrylate) is liquid and colourless.

Both reaction products were stored in the dark at 60° C. The urethane acrylate prepared by the process according to the invention was still liquid after two weeks at 60° C., without any significant change in the viscosity. The urethane acrylate based on IPDI with a high peroxide content was solid after 5 h at 60° C., and was therefore not storage stable.

1. A process for preparing at least one ethylenically unsaturated, urethane group-containing compound from
i) diisocyanate,
ii) at least one compound having at least one hydroxyl group and at least one ethylenically unsaturated, polymerizable group,
iii) and optionally at least one hydroxyl group-containing compound,
in which
1. diisocyanate is subjected to a determination of the peroxide content immediately prior to the reaction,
2. a) if the determined peroxide content is greater than or equal to 20 mmol/kg, the diisocyanate is subjected to distillative purification until the determined peroxide content is less than 20 mmol/kg, or
or
b) if the determined peroxide content is less than 20 mmol/kg, no distillative purification is performed, and
3. then i) distilled diisocyanate of a) and/or untreated diisocyanate of b) is reacted with ii) at least one compound having at least one hydroxyl group and at least one ethylenically unsaturated, polymerizable group and optionally iii) with at least one hydroxyl group-containing compound to give an ethylenically unsaturated, urethane group-containing compound.

2. The process according to claim 1, wherein the process uses a diisocyanate.

3. The process according to claim 1, wherein at least one of the diisocyanates used is a (cyclo)aliphatic diisocyanate.

4. The process according to claim 1, wherein at least one of the diisocyanates used is isophorone diisocyanate or 4,4′-diisocyanatodicyclohexylmethane.

5. The process according to claim 1, wherein the compound having at least one hydroxyl group and at least one ethylenically unsaturated group is selected from the group consisting of hydroxyalkyl acrylates, hydroxyalkyl methacrylates and hydroxyalkyl vinyl ethers.

6. The process according to claim 5, wherein the alkylene radicals of the hydroxyalkyl acrylates, hydroxyalkyl methacrylates or hydroxyalkyl vinyl ethers are C₂-C₁₀ alkylene radicals.

7. The process according to claim 1, wherein at least one hydroxyl group-containing compound iii) is used in the process.

8. The process according to claim 7, wherein the at least one hydroxyl group-containing compound iii) is selected from the group consisting of aliphatic diols, aliphatic polyols, polyester diols and polyester polyols, and polyether diols and polyether polyols.

9. The process according to claim 1, wherein the reaction is conducted in the presence of at least one catalyst at temperatures of 0-160° C.

10. The process according to claim 9, wherein the catalyst is selected from the group consisting of diazabicyclooctane, dibutyltin laurate, bismuth neodecanoate, zinc octoate and zirconium acetylacetonate.

11. The process according to claim 7, wherein the process is conducted such that first diisocyanate i) is reacted with at least one compound having at least one hydroxyl group and at least one ethylenically unsaturated, polymerizable group ii) and only thereafter is at least one hydroxyl group-containing compound iii) added.

12. The process according to claim 1, wherein in step 2.
a) if the determined peroxide content is greater than 10 mmol/kg, the diisocyanate is subjected to distillative purification until the determined peroxide content is less than or equal to 10 mmol/kg but greater than or equal to 0.1 mmol/kg,
or
if the determined peroxide content is less than 0.1 mmol/kg, peroxide is added until the peroxide content is greater than or equal to 0.1 mmol/kg but less than or equal to 10 mmol/kg, or
b) if the determined peroxide content is less than or equal to 10 mmol/kg but greater than or equal to 0.1 mmol/kg, no further action is taken.
